(19) United States
(12) Patent Application Publication
(10) Pub. No.: US 2025/0352194 A1
(43) Pub. Date: Nov. 20, 2025

(54) ENDOSCOPE CLIP USING SHAPE MEMORY ALLOY

(71) Applicant: KOREA UNIVERSITY RESEARCH AND BUSINESS FOUNDATION, Seoul (KR)

(72) Inventors: Kwang Dae HONG, Anyang-si (KR); Jeongho SOHN, Seoul (KR); Sunseok YOON, Ansan-si (KR)

(73) Assignee: KOREA UNIVERSITY RESEARCH AND BUSINESS FOUNDATION, Seoul (KR)

(21) Appl. No.: 19/285,984

(22) Filed: Jul. 30, 2025

Related U.S. Application Data
(63) Continuation of application No. PCT/KR2024/001552, filed on Feb. 1, 2024.

(30) Foreign Application Priority Data
Feb. 2, 2023 (KR) 10-2023-0014055
Feb. 1, 2024 (KR) 10-2024-0015554

(51) Int. Cl. A61B 17/00

(52) U.S. Cl. CPC A61B 17/0057; A61B 17/00234; A61B 2017/00004; A61B 2017/00296; A61B 2017/00477; A61B 2017/00584; A61B 2017/00592; A61B 2017/00623; A61B 2017/00862; A61B 2017/00867; A61B 2017/00951

(57) ABSTRACT

An endoscope clip using a shape memory alloy, according to an embodiment of the present invention, comprises a shape memory clip which is made of a shape memory alloy, and which is fixed to an edge portion thereof and restored to the original shape at a preset shape change temperature, wherein the shape memory clip includes a coupling part and pin parts vertically bent from the ends of the coupling part so as to be inserted into and fixed to the edge portion.

### CROSS-REFERENCE TO RELATED APPLICATION

Pursuant to 35 USC 120 and 365 (c), this application is a continuation of International Application No. PCT/KR2024/001552 filed on Feb. 1, 2024, and claims the benefit under 35 USC 119 (a) of Korean Application No. 10-2023-0014055 filed on Feb. 2, 2023 and Korean Application No. 10-2024-0015554 filed on Feb. 1, 2024, in the Korean Intellectual Property Office, the entire disclosure of which is incorporated herein by reference for all purposes.

### TECHNICAL FIELD

The present invention relates to an endoscope clip using a shape memory alloy.

### BACKGROUND ART

An endoscope is an instrument designed to insert a machine and observe organs of which lesions cannot be directly seen without surgery or autopsy. Generally, a typical endoscope includes a bronchoscope, esophagoscope, gastroscope, duodenoscope, proctoscope, cystoscope, and laparoscope, and other special types include a thoracoscope, mediastinoscope, and cardioscope.

The endoscope enters the body and various procedures such as inspection, diagnosis, sample collection, and containment can be performed using special tools along with an observation camera.

In particular, when suturing a perforation site, a procedure of suturing a perforation site may be performed after fixing a clip to the endoscope. However, there was a problem that a conventional clip was not applicable when a size of the perforation site was 1 cm or more or when the elasticity of the tissue was reduced due to inflammation.

Specifically, a conventional endoscope clip is mainly used for hemostasis and often fails to ligate when applied to perforation, and thus emergency surgery is required in most cases, and the conventional endoscope clip is opened at an angle of 120° to 150°, so that there was a problem that when the perforation is more than 1 cm or the tissue of the perforation is weak, they cannot be ligated.

In this case, Apollo surgery or OTSC is used, and Apollo surgery should be equipped with a separate device in the conventional endoscope, add another working port, and use a device specially attached to the tip, so that it has the disadvantage of requiring knowledge of the operating method and purchase of the devices.

In addition, since OTSC is mounted on the tip of the endoscope and has a saw-tooth structure that bites the defective area, it can strongly connect the tissue, but when the tissue is weak due to excessive force, there is a risk of tearing and it is difficult to apply to a defective area with a large area, and there is a disadvantage of requiring knowledge of a separate operating method.

### DISCLOSURE

### Technical Problem

The present invention is directed to providing an endoscope clip using a shape memory alloy that does not require the purchase of additional equipment other than a clip because the clip can be fixed to a conventional endoscope to suture.

In addition, the present invention is directed to providing an endoscope clip using a shape memory alloy that can be applied to a wide defective area or perforation area and enables suturing by selecting a suitable clip according to a size of the defective area.

Meanwhile, technical problems to be achieved in the present invention are not limited to the above-mentioned technical problems, and other technical problems not mentioned may be clearly understood by those having ordinary skill in the art from the following description.

### Technical Solution

An endoscope clip using a shape memory alloy according to an embodiment of the present invention includes: a shape memory clip which is made of a shape memory alloy, fixed to an edge portion thereof, and restored to its original shape at a preset shape change temperature; wherein the shape memory clip includes a coupling part and a pin part that is bent at an end of the coupling part to be inserted and fixed into the edge portion.

The shape memory clip is fixed to the edge portion thereof and may be restored to its original shape at the preset shape change temperature. The shape memory clip includes a coupling part, a bending part bent at a preset shape change angle at both ends of the coupling part, and a pin part that is bent at an end of the bending part to be inserted and fixed into the edge portion.

In the shape memory clip, the bending part may be bent in a point-symmetrical structure based on the coupling part at the both ends of the coupling part to form a “Z” shape.

In the shape memory clip, a pair of bending parts may be formed to be bent vertically with respect to the coupling part on a plane parallel to a longitudinal direction of the coupling part.

The pin part may be formed to protrude in a direction perpendicular to the plane from end portions of the pair of bending parts.

In addition, the endoscope clip using a shape memory alloy according to an embodiment of the present invention further includes a gripping part that is gripped by an endoscope and to which the shape memory clip is fixed.

In addition, the gripping part includes a body in which a fixing groove into which the shape memory clip is inserted and fixed is formed on one side surface thereof and a protrusion portion that protrudes on the other side surface of the body to be gripped by the endoscope.

In addition, the gripping part further includes an uneven portion protruding at least one on an outer surface of the protrusion portion to prevent slipping when gripping biopsy forceps of the endoscope.

In addition, an adhesive is applied on one side surface of the body, or the body is provided with a bioabsorbable material.

In addition, the coupling part and a bending portion of the pin part is restored to the original shape from a preset shape change angle to a preset original shape angle at the preset shape change temperature, and in the edge portion, fixed portions fixed into the pin part are pulled and narrowed together by restoring the original shape of the bending portion.

In addition, the coupling part and a bending portion of the bending part are restored to an original shape from the preset shape change angle to the preset original shape angle at the preset shape change temperature, and in the edge portion, fixed portions fixed into the pin part are pulled and narrowed together by restoring the original shape of the bending portion.

In addition, the endoscope clip further includes a cap provided at a tip of the endoscope, wherein the cap is open on both sides, one side thereof is coupled to the tip of the endoscope, and an insertion groove into which the gripping part and the shape memory clip may be inserted is formed on the other side thereof.

In addition, the shape memory clip has elasticity to be bent and inserted into the insertion groove.

In addition, in the cap, a fitting groove into which the pin part of the shape memory clip is fitted and fixed is formed on an inner wall of the insertion groove.

The cap may have a coupling hole formed on one side thereof so as to be coupled to the endoscope. The coupling hole may be formed eccentrically from a center of the cap so that the biopsy forceps of the endoscope is disposed at the center of the cap.

In addition, the preset shape change temperature is 40° C. to 45° C.

### Advantageous Effects

According to an embodiment of the present invention, an endoscope clip can be applied to cases where perforation occurs during upper gastrointestinal endoscopy, perforation occurs during colonoscopy, leakage occurs at the anastomosis site after upper gastrointestinal surgery, or leakage occurs at the anastomosis site after colon (colon and rectum) surgery and can be easily operated and fixed to a conventional endoscope to suture, and thus there is no need to purchase additional equipment other than the clip.

In addition, according to an embodiment of the present invention, an endoscope clip can be applied to a wide defect area or perforation area, enables suturing by selecting a suitable clip according to a size of the defect area, and allows conservative treatment without additional surgery.

In addition, according to an embodiment of the present invention, it is possible to reduce the possibility of long-term treatment and death due to emergency surgery for patients who are elderly and have dangerous concomitant disease.

Meanwhile, effects obtainable from the present invention are not limited to the above-mentioned effects, and other effects not mentioned can be clearly understood those having ordinary skill in the art from the following description.

### DESCRIPTION OF DRAWINGS

FIGS. 1 to 4 are perspective views showing an endoscope clip using a shape memory alloy according to an embodiment of the present invention.

FIG. 5 is a view showing a state in which an endoscope clip using a shape memory alloy according to an embodiment of the present invention is fixed to an edge portion.

FIG. 6 is a perspective view showing an endoscope clip using a shape memory alloy according to an embodiment of the present invention.

FIG. 7, FIG. 8A, FIG. 8B, and FIG. 8C are views showing a process of restoring a shape memory clip of an endoscope clip using a shape memory alloy according to an embodiment of the present invention.

FIG. 9 is a view describing a preset shape change angle of a Z shape.

FIG. 10 and FIG. 11 are views showing a state in which an endoscope clip using a shape memory alloy according to an embodiment of the present invention is fixed to an edge portion.

FIGS. 12 to 14 are perspective views showing an endoscope clip using a shape memory alloy according to an embodiment of the present invention.

### MODES OF THE INVENTION

Hereinafter, the embodiments of the present invention will be described in more detail with reference to the attached drawings. The embodiments of the present invention can be modified in various forms, and the scope of the present invention should not be construed as being limited to the embodiments below. The embodiments are provided to more completely describe the present invention to those having ordinary skill in the art. Therefore, the shapes of elements in the drawings are exaggerated to emphasize a clearer description.

In order to clearly describe the solution to the problem to be solved by the present invention, the composition of the invention will be described in detail based on a preferred embodiment of the present invention with reference to the attached drawings. In assigning reference numerals to the components in the drawings, like reference numerals are assigned to like components even if they are in different drawings, and it is to be noted in advance that components in other drawings may be cited when necessary when describing the drawings.

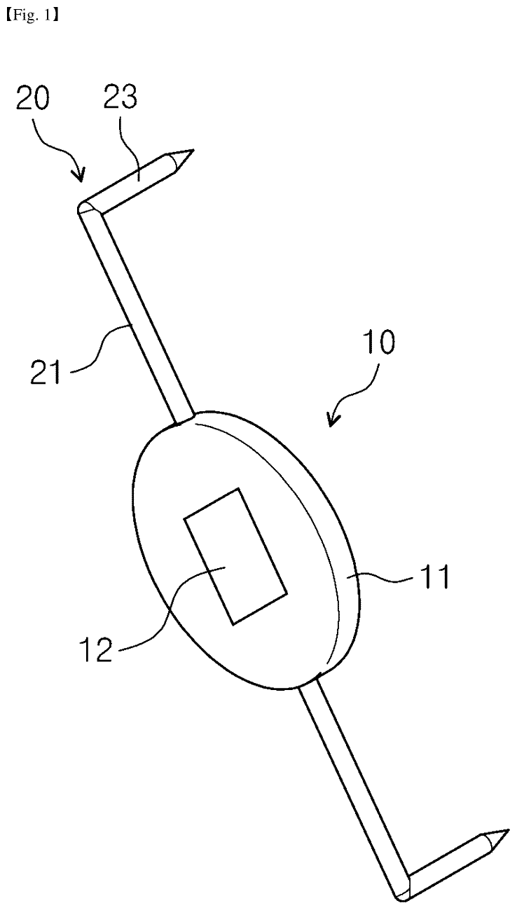

Referring to FIGS. 1 to 6, an endoscope clip using a shape memory alloy according to an embodiment of the present invention may include a gripping part 10 and a shape memory clip 20.

First, the gripping part 10 may be gripped by an endoscope.

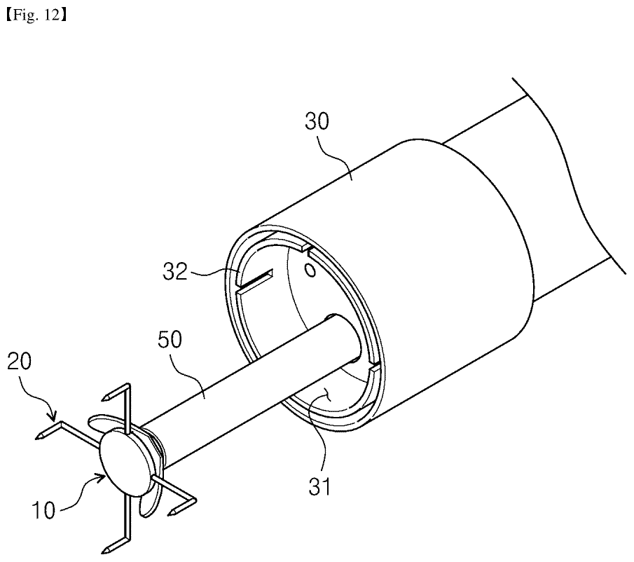

Here, the endoscope may be composed of a channel that is inserted into a body, biopsy forceps (element number 50 of FIG. 12) provided at a tip of an insertion part inside the endoscope, and an operating unit for operating the biopsy forceps 50, but it is not limited thereto. In addition, since the endoscope is a known technology, a detailed description thereof will be omitted.

The gripping part 10 may include a body 11, a protrusion portion 12, and an uneven portion 13.

The body 11 of the gripping part 10 may be gripped by the biopsy forceps 50.

In addition, a fixing groove 14 into which the shape memory clip 20 is inserted and fixed is formed on one side surface of the body 11.

The protrusion portion 12 may be gripped by the biopsy forceps 50. The protrusion portion 12 may protrude on the other side surface of the body 11 as shown in FIG. 1 and FIG. 6.

As another example, although not shown, the protrusion portion 12 may also be formed to be curved in an inward direction of the body from the other side of the body 11. In this case, the protrusion portion 12 may be formed as a plurality of recesses on the other side surface of the body 11, and the biopsy forceps 50 may be inserted into the protrusion portion 12 formed as the recess and the protrusion portion 12 may be gripped by the biopsy forceps 50.

Referring to FIG. 6, the uneven portion 13 may protrude at least one on an outer surface of the protrusion portion 12 to prevent slipping when gripped by the biopsy forceps 50 of the endoscope.

Meanwhile, an adhesive may be applied on one side surface of the body 11, or the body 11 may be provided with a bioabsorbable material.

That is, one side surface of the body 11 is a surface that is in contact with skin tissue, and when the shape memory clip 20, which will be described later, pulls the edge portion 40, a defective area 41 is ligated, and one side surface of the body 11 may be in contact with the defective area 41.

In this case, the adhesive applied on the body 11 may be fixed to the edge portion 40 not only to cover and protect the defective area 41, but also to prevent the defective area 41 from being opened. In addition, the bioabsorbable material applied on the body 11 may be fixed to the edge portion 40 not only to cover and protect the defective area 41, but also to promote regeneration of tissue.

In this case, the bioabsorbable material may include thrombin and fibrinogen.

Accordingly, the gripping part 10 may be easily gripped by the biopsy forceps 50 of the endoscope and may be fixed to the defective area 41 and the edge portion 40.

The shape memory clip 20 may be coupled to the gripping part 10, made of a shape memory alloy, and fixed to the edge portion 40 thereof to be restored to its original shape at a preset shape change temperature.

Here, the shape memory clip 20 is made of the shape memory alloy, and the shape memory alloy may be formed in a coil spring structure, but it is not limited thereto.

Since the shape memory alloy has unique properties which are a shape memory effect and superelasticity, the shape memory alloy with the coil spring-like form may be used in various fields such as medical devices, actuators, sensors, and robotics.

In addition, the shape memory alloy with the coil spring form may utilize characteristics of returning to its original shape when heat is applied. For example, the shape memory alloy may be used in a medical stent in which a shape change occurs due to body temperature or heat applied from outside, an actuator that mimics muscle movements, and a temperature sensor, etc. The shape memory alloy with this form has characteristics of decreasing or expanding in length when heat is applied, and thus it is useful for controlling precise force and movement.

Accordingly, the shape memory clip 20 of the present invention may be formed of a cylindrical or a circular pillar shaped shape memory alloy and may be formed in the coil spring structure, but it is not limited thereto.

In the present invention, the shape memory clip 20 may be composed of a plurality of embodiments.

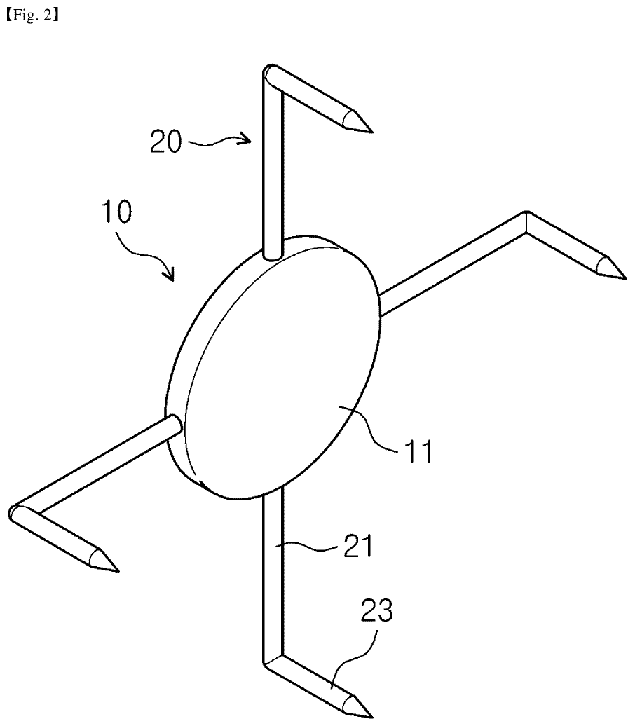

Referring to FIG. 1 and FIG. 2, the embodiments of the shape memory clip 20 may include a coupling part 21 and a pin part 23.

The coupling part 21 is coupled to the body 11 of the gripping part 10. In addition, the coupling part 21 may be provided in an integral form at the body 11 of the gripping part 10, but it is not limited thereto.

In this case, the coupling part 21 may be coupled on a plane of the body 11 or coupled on a plane parallel to the plane of the body 11. In addition, the plane of the body 11 refers to a plane that is parallel to one side surface or the other side surface.

The pin part 23 may be vertically bent from an end of the coupling part 21 to be inserted and fixed into the edge portion 40.

In this case, the pin part 23 may be bent vertically from the plane of the body 11 of the gripping part 10.

Here, the coupling part 21 may be provided to protrude in an outward direction based on the body 11 and may be provided in two or more. For example, the coupling part 21 may be provided in two as in FIG. 1 or in four as in FIG. 2.

In addition, the pin part 23 is provided to correspond to the coupling part 21, and thus the pin part 23 may be provided in two or more.

In this case, the coupling part 21 and the bending part 22 of the pin part 23 may be restored to a preset original shape angle at a preset shape change temperature.

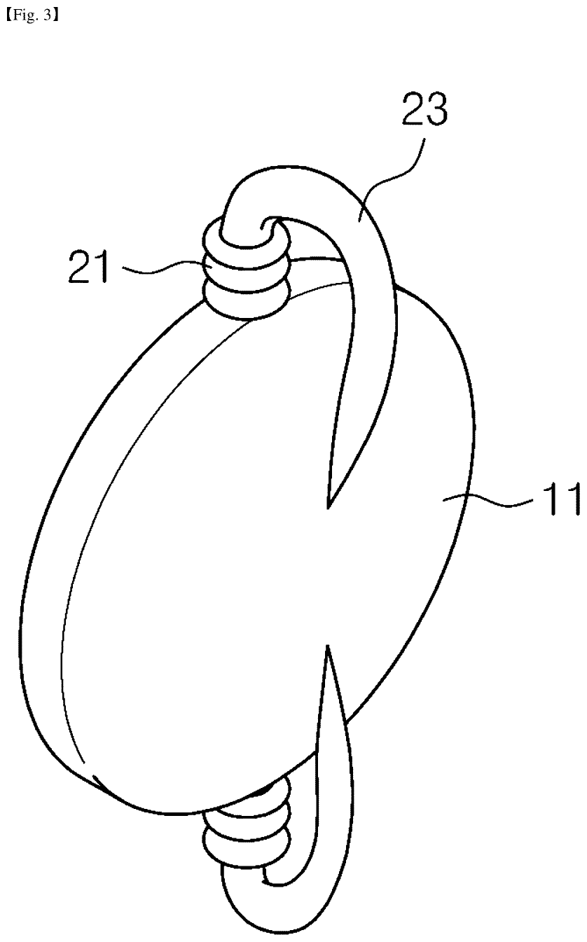

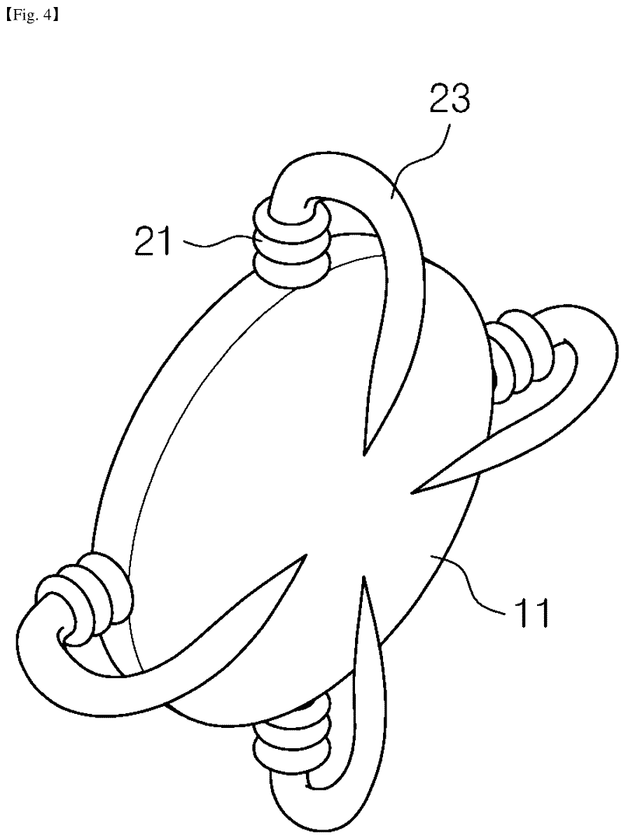

For example, as in FIG. 3 and FIG. 4, the coupling part 21 and the bending part 22 of the pin part 23 may be restored to the original shape by bending the pin part 23 to a preset original shape angle toward one side surface of the body 11 at the preset shape change temperature.

Accordingly, in the edge portion 40, fixed portions fixed into the pin part 23 may be pulled and narrowed together by restoring the original shape of the bending part 22.

For example, a defective area 41 such as a perforation may be narrowed or sutured in contact with each other as the bending part 22 of the shape memory clip 20 fixed to the edge portion 40 is restored to its original shape at the preset shape change temperature. In this case, the coupling part 21 may be provided in four, but it is not limited thereto.

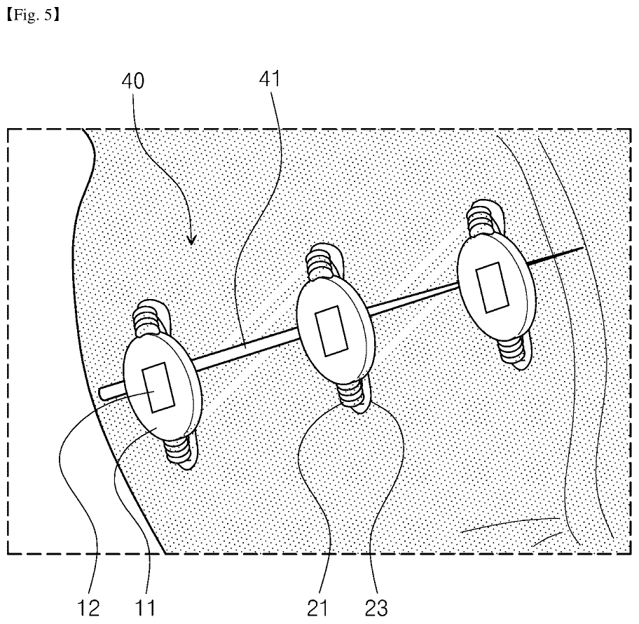

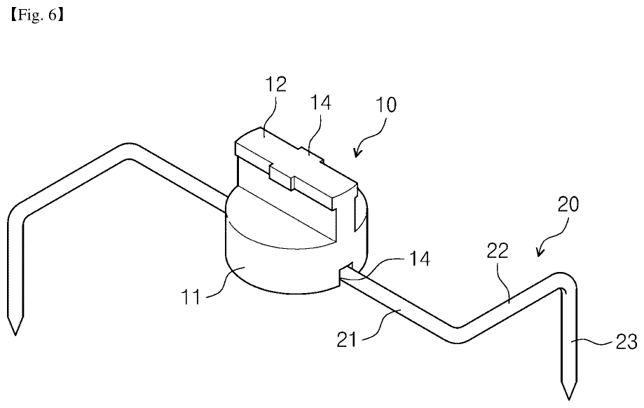

As another example, as shown in FIG. 5, in a state in which a plurality of the shape memory clips 20 are fixed to the edge portion 40 in an incision direction, the incised defective area 41 may be sutured as the shape memory clips 20 are restored to their original shape at the preset shape temperature.

Referring to FIG. 6, in another embodiment of the shape memory clip 20, the shape memory clip 20 may include a coupling part 21, a bending part 22, and a pin part 23.

The coupling part 21 is coupled to the body 11 of the gripping part 10. In this case, the coupling part 21 may be fitted and fixed into the fixing groove 14 of the body 11.

The bending part 22 is bent at a preset shape change angle at both ends of the coupling part 21. In this case, the bending part 22 may be bent to a plane identical to a plane of a longitudinal direction of the coupling part 21. That is, in the shape memory clip 20, a pair of the bending parts 22 may be bent in a point-symmetrical structure based on the coupling part 21 at the both ends of the coupling part 21 to form a “Z” shape.

In the shape memory clip 20, the pair of bending parts 22 may be formed to be bent vertically with respect to the coupling part 21 on a plane parallel to the longitudinal direction of the coupling part 21. The coupling part 21 and the bending part 22 may be in close contact with skin tissue. The pin part 23 may be formed to protrude in a direction perpendicular to a plane on which the coupling part 21 and the bending part 22 are formed from end portions of the pair of bending parts 22.

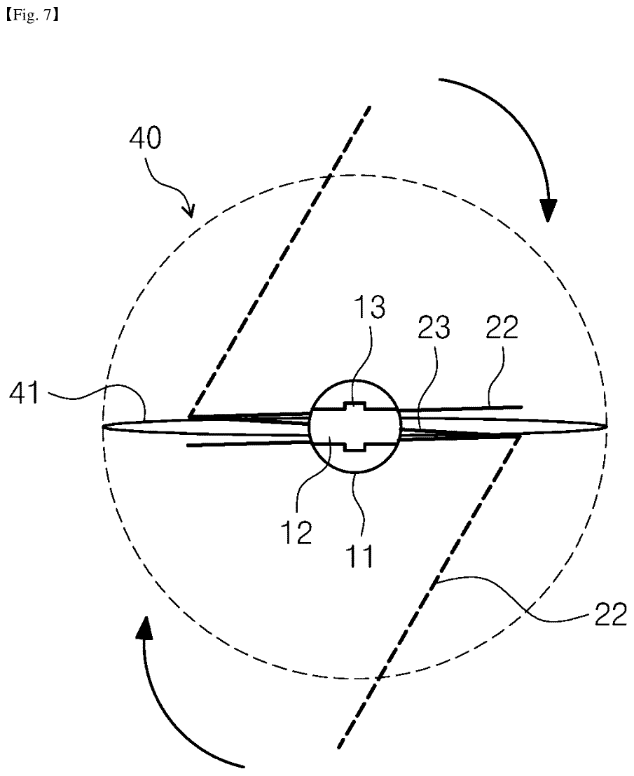

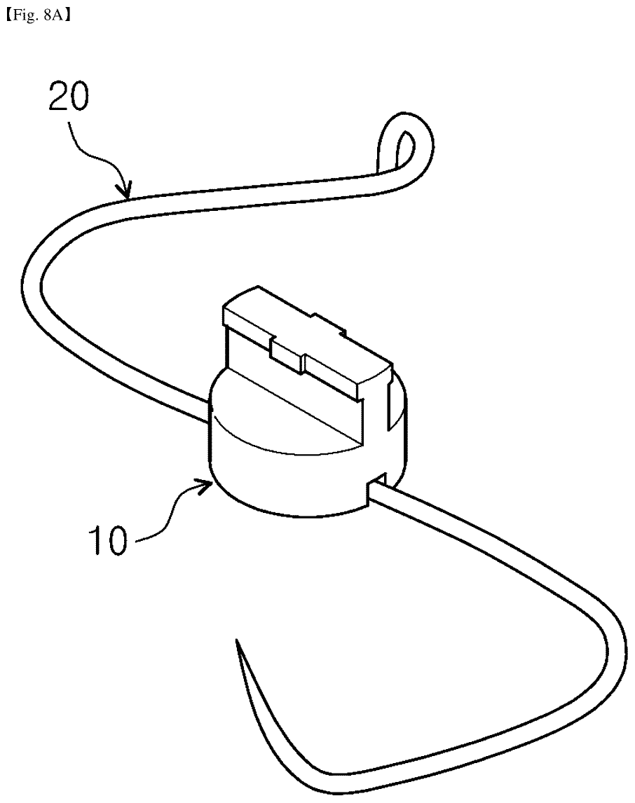

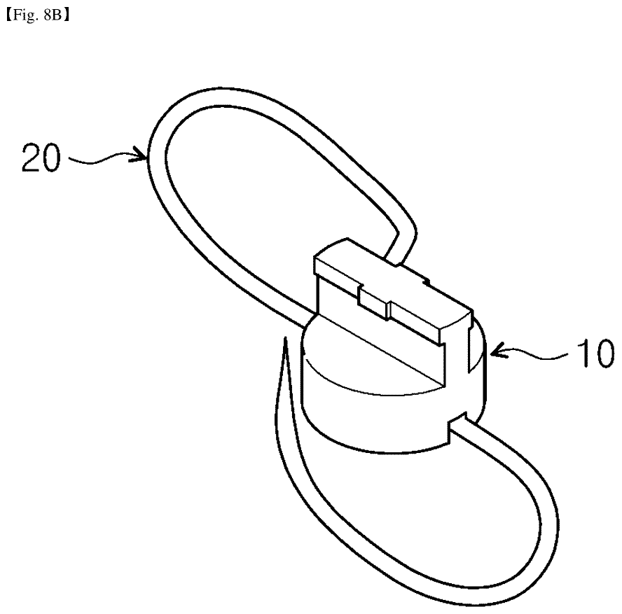

In addition, referring to FIG. 7 and FIGS. 8A to 8C, shape change of the shape memory clip 20 with the Z shape may be identified. That is, as the bending parts 22 are formed on the same plane as the coupling part 21 and bending is performed on a plane in which a bending direction is the same, the edge portion 40 may be pulled and the perforation or incision area may be sutured.

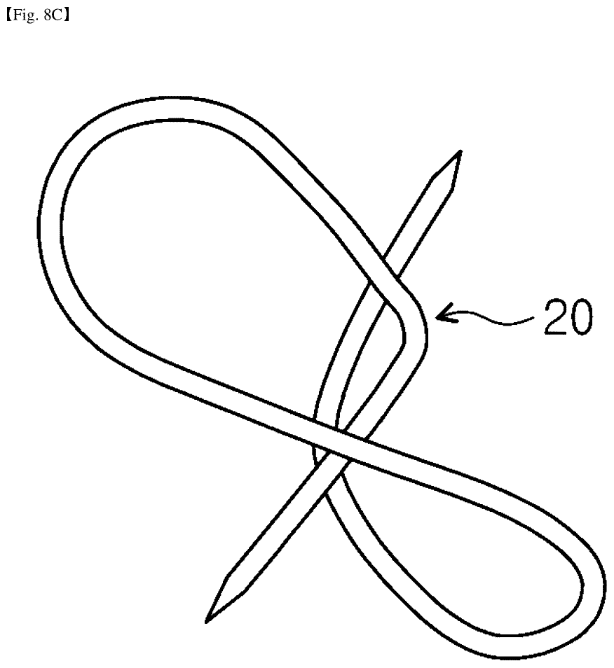

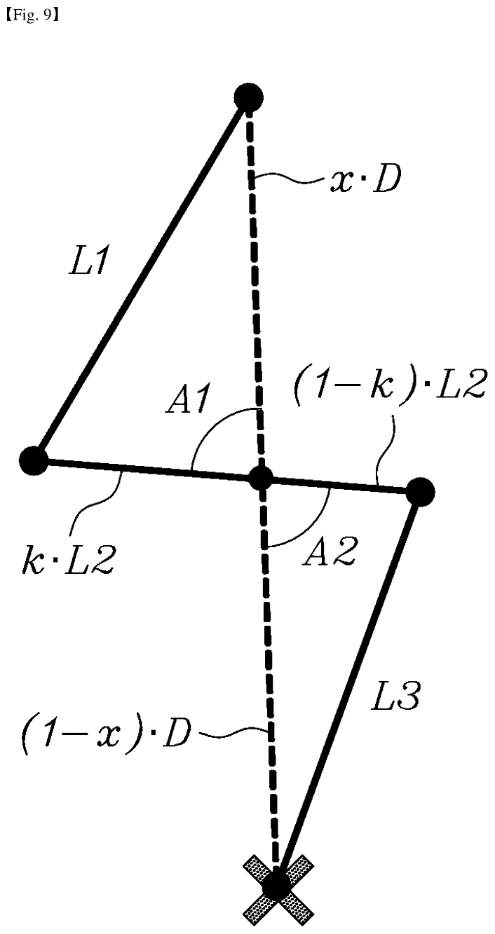

In addition, referring to FIG. 9, the coupling part 21 may have a length of L₂, and the bending parts 22 may have lengths of L₁ and L₃.

In this case, an angle of the Z shape may be determined according to [Equation 1] to [Equation 5] below. That is, the preset shape change angle at which the coupling part 21 and the bending parts 22 are bent to each other may be determined according to [Equation 1] to [Equation 5] below.

$$cos ( {A}_{1} ) = \frac{{( x \cdot D )}^{2} + {( k \cdot {L}_{2} )}^{2} - {L}_{1}^{2}}{2 \cdot ( x \cdot D ) \cdot ( k \cdot {L}_{2} )} \qquad [ Equation 1 ]$$

$$cos ( {A}_{2} ) = \frac{{( ( 1 - x ) \cdot D )}^{2} + {( ( 1 - k ) \cdot {L}_{2} )}^{2} - {L}_{3}^{2}}{2 \cdot ( ( 1 - x ) \cdot D ) \cdot ( ( 1 - k ) \cdot {L}_{2} )} \qquad [ Equation 2 ]$$

[Equation 1] and [Equation 2] may use the cosine law to calculate angle A₁ and A₂.

Here, x and k are variables representing a ratio, and D, L₁ and L₂ represent distance and length, respectively. Also, it is assumed that A₁=A₂.

$$\frac{{( x \cdot D )}^{2} + {( k \cdot {L}_{2} )}^{2} - {L}_{1}^{2}}{2 \cdot ( x \cdot D ) \cdot ( k \cdot {L}_{2} )} = \frac{{( ( 1 - x ) \cdot D )}^{2} + {( ( 1 - k ) \cdot {L}_{2} )}^{2} - {L}_{3}^{2}}{2 \cdot ( ( 1 - x ) \cdot D ) \cdot ( ( 1 - k ) \cdot {L}_{2} )} \qquad [ Equation 3 ]$$

[Equation 3] is a result obtained by using a fact that cosine values of A₁ and A₂ are the same and it is possible because the two angles are the same. Values of x and k may be found through [Equation 3].

$${L}_{1}^{2} \cdot ( - ( 1 - x ) ( 1 - k ) ) + {L}_{2}^{2} \cdot ( - {k}^{3} + {k}^{2} - xk + {xk}^{2} ) + {L}_{3}^{2} \cdot ( xk ) + {D}^{2} \cdot ( - {x}^{3} + {x}^{2} - xk + {x}^{2} k ) = 0 \qquad [ Equation 4 ]$$

[Equation 4] is an equation that is set to 0 by rearranging and simplifying previous equations. A goal is to find the value of x through this equation. Here, an equation is set to find a solution by rearranging relationships between the variables.

$$E ( x ) = {L}_{1}^{2} \cdot ( - ( 1 - x ) ( 1 - k ) ) + {L}_{2}^{2} \cdot ( - {k}^{3} + {k}^{2} - xk + {xk}^{2} ) + {L}_{3}^{2} \cdot ( xk ) + {D}^{2} \cdot ( - {x}^{3} + {x}^{2} - xk + {x}^{2} k ) \qquad [ Equation 5 ]$$

[Equation 5] is the expression of [Equation 4] as a function E(x). When the x value for which this function becomes 0 is found, the preset shape change angle of the Z shape may be calculated.

The above-described Equations are equations for solving an inverse kinematics problem of the Z shape.

Here, the variables x and k may be set to 0.5. Accordingly, by calculating the Equation, a value of E(x) may be obtained, and the lengths of L₁, L₂, and L₃ may be set.

As described above, the variables may be used to create a geometric model in which two triangles may dynamically change while maintaining a constant angle.

The pin part 23 may be bent at an end of the bending part 22 to be inserted and fixed into the edge portion 40.

In this case, the pin part 23 may be bent vertically from the plane of the body 11 of the gripping part 10.

Here, referring to FIGS. 8A to 8C, the coupling part 21 and the bending portion of the bending part 22 may be restored to the original shape from the preset shape change angle to the preset original shape angle at the preset shape change temperature.

In addition, in the edge portion 40, fixed portions fixed into the pin part 23 are pulled and narrowed together by restoring the original shape of the bending part 22.

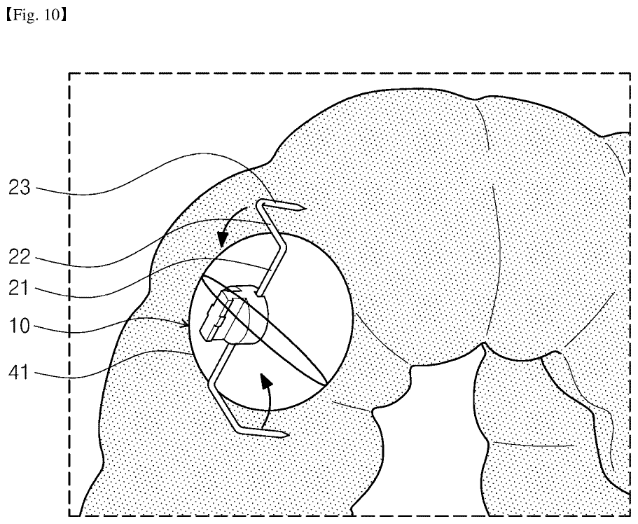

For example, referring to FIG. 7, FIGS. 8A to 8C, FIG. 10, and FIG. 11, the defective area 41 such as the perforation or the incision area is narrowed as the bending part 22 of the shape memory clip 20 fixed to the edge portion 40 is restored to its original shape at the preset shape change temperature. In this case, as a separation distance of the pin part 23 on the plane becomes closer, the defective areas 41 may be sutured substantially in contact with each other. In this case, one side surface of the body 11 may cover and suture an untouched defective area 41.

Meanwhile, in order to model a heat transfer problem for the shape memory clip 20 in the Z shape, following Equations may be used.

Variables described in [Equation 6] to [Equation 10] below are as follows: T represents temperature, t represents time, x, y, and z represent three-dimensional spatial coordinate, r represents a radius in a cylindrical coordinate system, κ (kappa) represents thermal diffusivity, Δt represents a time interval used in a finite-difference method, Δx represents a gap dividing a space in the finite-difference method, Q′ (Q point) represents a heat generation rate per unit volume, ρ (rho) represents density, and c represents specific heat.

$$\frac{\partial T}{\partial t} = κ \frac{{\partial }^{2} T}{\partial {x}^{2}} . \qquad [ Equation 6 ]$$

Here, T represents temperature, t represents time, x represents spatial coordinate, and κ represents thermal diffusivity.

[Equation 6] represents a one-dimensional heat conduction equation and shows that a temperature change over time is proportional to a second differential of a spatial temperature change. That is, it may be used to describe how heat is transmitted through the inside of the material.

$${T}_{i , j + 1} = {T}_{i , j} + κ \frac{\partial t}{\partial {x}^{2}} [ {T}_{i - 1 , j} - 2 {T}_{i , j} + {T}_{i + 1 , j} ] \qquad [ Equation 7 ]$$

[Equation 7] is a modified equation using the finite-difference method, and it is possible to calculate linear heat flow.

$$\frac{\partial T}{\partial t} = κ ( \frac{{\partial }^{2} T}{\partial {r}^{2}} + \frac{1}{r} \frac{\partial T}{\partial r} ) . \qquad [ Equation 8 ]$$

[Equation 8] is an equation representing heat transfer in the cylindrical coordinate system.

$${T}_{i , j + 1} = {T}_{i , j} + κ \frac{\partial t}{2 i δ {x}^{2}} \times [ ( 2 i + 1 ) {T}_{i + 1 , j} - 4 {iT}_{i , j} + ( 2 i - 1 ) {T}_{i - 1 , j} ] . \qquad [ Equation 9 ]$$

[Equation 9] is a modification of the finite-difference method for calculating heat flow in the cylindrical coordinate system.

$$\frac{\partial T}{\partial t} = κ ( \frac{{\partial }^{2} T}{\partial {x}^{2}} + \frac{{\partial }^{2} T}{\partial {y}^{2}} + \frac{{\partial }^{2} T}{\partial {z}^{2}} ) + \frac{\dot{Q}}{ρ c} \qquad [ Equation 10 ]$$

[Equation 10] represents heat transfer in a three-dimensional space, and it is possible to consider a presence of a heat source.

Meanwhile, the preset shape change temperature (temperature at which the shape memory clip returns to its original shape) may be 40 to 45° C., but it is not limited thereto.

For example, in a case in which the temperature is lower than 40° C., a rate of shape change according to the body temperature may occur slowly immediately after application to skin tissue.

In order to increase the rate of shape change, a method of firing heated water (higher temperature water may be injected as heat loss may occur when water moves through the endoscope based on 40 to 45° C. when coming out of a port) and/or a method of heating by applying electricity may be used, but it is not necessarily limited thereto.

In addition, the endoscope is connected to carbon dioxide gas, and thus the shape memory clip 20 is not transformed by the body temperature while moving inside the body.

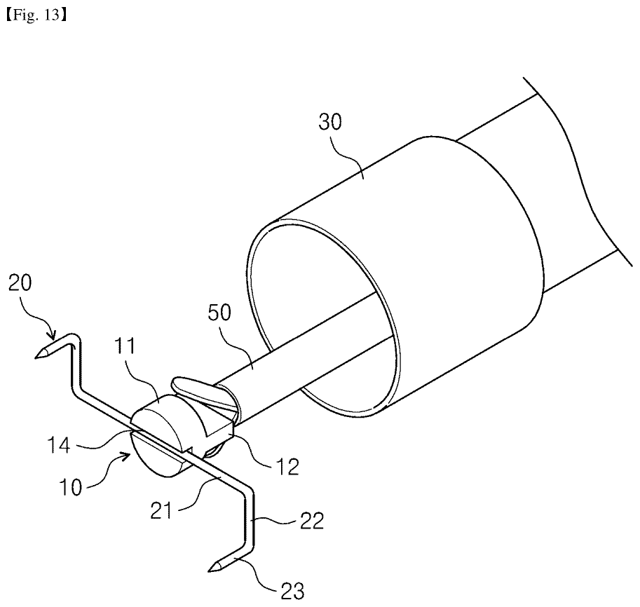

Meanwhile, referring to FIGS. 12 to 14, the endoscope clip using a shape memory alloy according to the present invention may further include a cap 30.

The cap 30 is provided at a tip of the endoscope. The cap 30 may be formed of a transparent material. Accordingly, it is possible to prevent securing a visual field from being obstructed by light being blocked by the cap 30.

In addition, the cap 30 is open on both sides, one side thereof is coupled to the tip of the endoscope, and an insertion groove 31 into which the gripping part 10 and the shape memory clip 20 may be inserted is formed on the other side thereof.

Here, the shape memory clip 20 has elasticity to be bent and inserted into the insertion groove 31. In this case, the shape memory clip 20 may be fixed to the cap 30.

That is, the shape memory clip 20 is fixed to the cap 30 to be moved inside the body together with the endoscope.

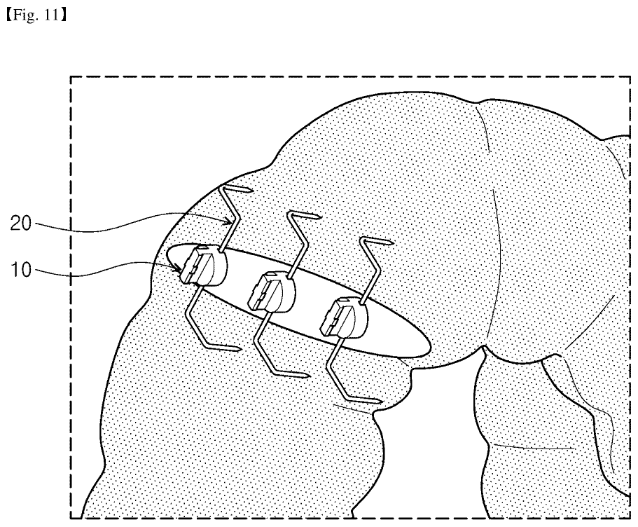

In addition, in the cap 30, a fitting groove 32 into which the pin part 23 of the shape memory clip 20 is fitted and fixed is formed on an inner wall of the insertion groove 31 as in FIG. 11. Through this, the shape memory clip 20 may be more stably fixed to the cap 30.

In addition, the cap 30 may be formed in a cylindrical shape. In this case, a communication groove provided to penetrate the wall surface of the cap 30 may be formed on a wall surface of the cap 30 with the cylindrical shape. Specifically, a plurality of the communication grooves may be provided to form a communication between the inside of the body and the insertion groove 31.

Accordingly, it is possible to prevent light that reaches the inside of the body from being blocked by the wall surface of the cap 30.

In this case, the cap 30 may have a coupling hole 33 formed on one side thereof so as to be coupled to the endoscope.

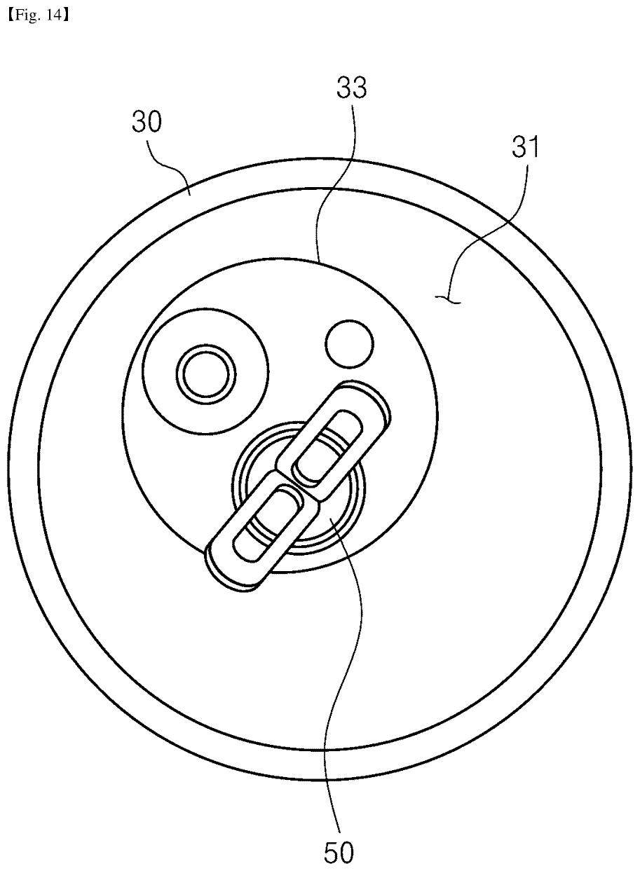

Referring to FIG. 14, the coupling hole 33 may be formed at a center of the cap 30 or eccentrically therefrom. Accordingly, the endoscope may be provided at the center of the cap 30 or eccentrically therefrom.

The coupling hole 33 is formed eccentrically because the biopsy forceps 50 of the endoscope may not be positioned at a center of a channel. That is, it is preferable for the coupling hole 33 to be formed eccentrically so that the biopsy forceps 50 are positioned at the center of the cap 30. When the coupling hole 33 is not formed eccentrically, a working port may also grip the shape memory clip 20.

In addition, the cap 30 mounted on the tip of the endoscope may be manufactured by a 3D printer using a thermoplastic polyurethane (TPU) material and may also be manufactured by a stereolithography (SLA) printer using thermosetting resin, but it is not limited thereto.

The above detailed description is illustrative of the present invention. In addition, the foregoing content describes the preferred embodiments of the present invention, and the present invention can be used in various other combinations, changes, and environments. That is, changes or modifications are possible within the scope of the concept of the invention disclosed herein, the scope equivalent to the described disclosure, and/or the scope of technology or knowledge in the art. The described embodiments are intended to describe the best possible state for implementing the technical idea of the present invention, and various changes required for specific application fields and uses of the present invention are also possible. Accordingly, the above detailed description of the invention is not intended to limit the invention to the disclosed embodiments. In addition, the appended claims should be construed to include other embodiments.

1. An endoscope clip using a shape memory alloy comprising:
a shape memory clip which is made of a shape memory alloy, fixed to an edge portion thereof, and restored to its original shape at a preset shape change temperature;
wherein the shape memory clip includes a coupling part and a pin part that is bent at an end of the coupling part to be inserted and fixed into the edge portion.

2. The endoscope clip using a shape memory alloy of claim 1, wherein the shape memory clip further includes a bending part bent at a preset shape change angle at both ends of the coupling part.

3. The endoscope clip using a shape memory alloy of claim 2, wherein, in the shape memory clip, the bending part is bent in a point-symmetrical structure based on the coupling part at the both ends of the coupling part to form a “Z” shape.

4. The endoscope clip using a shape memory alloy of claim 3, wherein, in the shape memory clip, a pair of bending parts are formed to be bent vertically with respect to the coupling part on a plane parallel to a longitudinal direction of the coupling part, and
the pin part is formed to protrude in a direction perpendicular to the plane from end portions of the pair of the bending parts.

5. The endoscope clip using a shape memory alloy of claim 2, further comprising a gripping part that is gripped by an endoscope and to which the shape memory clip is fixed.

6. The endoscope clip using a shape memory alloy of claim 5, wherein the gripping part includes a body in which a fixing groove into which the shape memory clip is inserted and fixed is formed on one side surface thereof and
a protrusion portion formed on the other side surface of the body to be gripped by the endoscope.

7. The endoscope clip using a shape memory alloy of claim 6, wherein the gripping part further includes an uneven portion protruding at least one on an outer surface of the protrusion portion to prevent slipping when gripped by the endoscope.

8. The endoscope clip using a shape memory alloy of claim 6, wherein an adhesive is applied on one side surface of the body, or the body is provided with a bioabsorbable material.

9. The endoscope clip using a shape memory alloy of claim 1, wherein the coupling part and a bending portion of the pin part is restored to the original shape from a preset shape change angle to a preset original shape angle at the preset shape change temperature, and
in the edge portion, fixed portions fixed into the pin part are pulled and narrowed together by restoring the original shape of the bending portion.

10. The endoscope clip using a shape memory alloy of claim 2, wherein the coupling part and a bending portion of the bending part are restored to an original shape from the preset shape change angle to the preset original shape angle at the preset shape change temperature, and
in the edge portion, fixed portions fixed into the pin part are pulled and narrowed together by restoring the original shape of the bending portion.

11. The endoscope clip using a shape memory alloy of claim 2, further comprising a cap provided at a tip of the endoscope,
wherein the cap is open on both sides, one side thereof is coupled to the tip of the endoscope, and an insertion groove into which the shape memory clip may be inserted is formed on the other side thereof.

12. The endoscope clip using a shape memory alloy of claim 11, wherein the shape memory clip has elasticity to be bent and inserted into the insertion groove.

13. The endoscope clip using a shape memory alloy of claim 11, wherein, in the cap, a fitting groove into which the pin part of the shape memory clip is fitted and fixed is formed on an inner wall of the insertion groove.

14. The endoscope clip using a shape memory alloy of claim 11, wherein the cap has a coupling hole formed on one side thereof so as to be coupled to the endoscope, and
the coupling hole is formed eccentrically from a center of the cap so that the biopsy forceps of the endoscope is disposed closer to the center of the cap than an outer periphery of the cap.

15. The endoscope clip using a shape memory alloy of claim 2, wherein the preset shape change temperature is 40° C. to 45° C.
